(19) United States
(12) Patent Application Publication
(10) Pub. No.: US 2025/0368676 A1
(43) Pub. Date: Dec. 4, 2025

(54) PROCESS FOR PREPARING NAMODENOSON

(71) Applicant: CAN-FITE BIOPHARMA LTD., Petah Tikva (IL)

(72) Inventors: Xiang Yang YU, Acton, MA (US); Dai KUANGCHU, Shanghai (CN)

(21) Appl. No.: 19/074,819

(22) Filed: Mar. 10, 2025

(30) Foreign Application Priority Data
May 31, 2024 (CN) 2024106944693

(51) Int. Cl. C07H 19/167

(52) U.S. Cl. CPC C07H 19/167

(57) ABSTRACT

The present invention provides a process for preparing namodenoson. More specifically, the present disclosure provides a simple and high yield process for Good Manufacturing Practice of namodenoson. The process involves the steps of protection, oxidation, chlorination, amination, reaction and final deprotection.

### TECHNICAL FIELD

The present subject matter relates to a process for the preparation of a highly specific and selective agonist at the A3 adenosine receptor (A3AR) known as namodenoson.

### REFERENCES

The following are references considered to be of relevance as a general background art to this disclosure:
Cohen S., et al., J. Cell Physiol. 2011; 226:2438-2447
Stemmer S. M., et al. Oncologist. 2013; 18:25-26
U.S. Pat. No. 6,790,839
U.S. Pat. No. 7,141,553
U.S. Pat. No. 7,589,075
U.S. Pat. No. 8,987,228
WO 2007/063538
WO 2009/050707
WO 2013/111132
WO 2017/090036

### BACKGROUND

Namodenoson, 2-Chloro-N⁶-(3-iodobenzyl)-adenosine-5′-N-methyluronamide, also known in the scientific literature as Cl-IB-MECA, is a highly selective orally bioavailable A3AR agonist. Namodenoson is effective in the treatment of cancer (U.S. Pat. No. 6,790,839 and WO 2013/111132), inflammatory diseases (U.S. Pat. Nos. 7,141,553, 8,987,228, WO 2007/063538), inhibiting viral replication (U.S. Pat. No. 7,589,075), inhibiting hepatocyte proliferation (WO 2009/050707), reducing ectopic fat accumulation (WO 2017/090036), inducing an apoptotic effect towards HCC in syngeneic orthotopic and xenograft experimental animal models (Cohen S., et al., J. Cell Physiol. 2011; 226:2438-2447) and others.

In an open-label phase I/II trial, the safety and efficacy of namodenoson were assessed in patients with advanced unresectable HCC, 67% of whom failed prior sorafenib treatment. Median overall survival (OS) was 7.8 months for the whole study population, and for Child-Pugh B patients (28%), it was 8.1 months. Namodenoson was safe and well-tolerated, and a direct correlation was found between A3AR expression levels at baseline and tumor response to namodenoson (Stemmer S. M., et al. Oncologist. 2013; 18:25-26).

### SUMMARY

The present disclosure concerns a process for preparing namodenosone of the following formula (I)

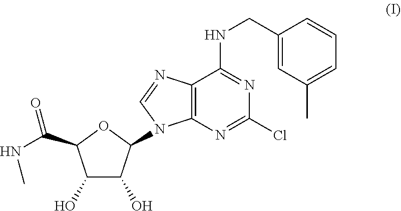 [SMILES: CNC(=O)[C@H]1O[C@@H](n2cnc3c(NCc4cccc(C)c4)nc(Cl)nc32)[C@H](O)[C@@H]1O]

which is suitable for large scale production, for example, production under Good Manufacturing Practice (GMP). The process of this disclosure, which comprise the steps of protection, oxidation, chlorination, amination, reaction, and final deprotection, as further discussed below.

### DETAILED DESCRIPTION

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

The term “a” or “an” as used herein includes the singular and the plural, unless specifically stated otherwise. Therefore, the terms “a,” “an,” or “at least one” can be used interchangeably in this application.

For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.” Accordingly, unless indicated to the contrary, the numerical parameters set forth in the following specification and claims are approximations that may vary depending upon the desired properties sought to be obtained. At the very least, each numerical parameter should at least be construed in light of the number of reported significant digits and by applying ordinary rounding techniques. In this regard, the term “about” denotes a quantity which may deviate (namely being higher or lower) by up to 10%, 15%, 20%, 25% or even 30%, from the stated quantity. For example, about 10 should be understood to be in the range of 9-11, 8.5-11.5, 8-12, 7.5-12.5, or even 7-13. Even where a value is given without the “about” qualification, these should be construed to mean to be about the indicated value, namely the value with a possible deviation as noted in this paragraph.

### Process for Preparing Namodenoson

The present subject matter provides a process for preparing namodenoson of formula (I)

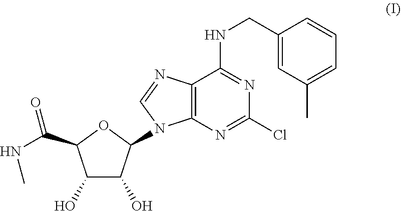 [SMILES: CNC(=O)[C@H]1O[C@@H](n2cnc3c(NCc4cccc(C)c4)nc(Cl)nc32)[C@H](O)[C@@H]1O]

the process comprises the steps of:
(a) protecting the hydroxyl groups of the diol of 2,6-dichloropurine riboside of formula (II)

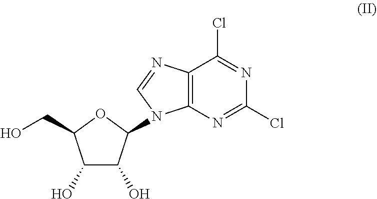 [SMILES: OC[C@H]1O[C@@H](n2cnc3c(Cl)nc(Cl)nc32)[C@H](O)[C@@H]1O]

to obtain a compound of formula (III);

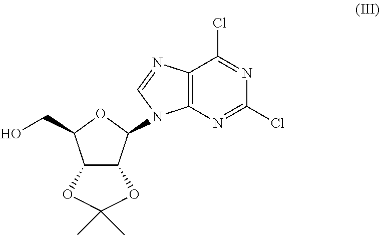 [SMILES: CC1(C)O[C@@H]2[C@H](O1)[C@@H](CO)O[C@H]2n1cnc2c(Cl)nc(Cl)nc21]

(b) oxidizing the compound of formula (III) in the presence of an oxidizing agent to obtain a compound of formula (IV);

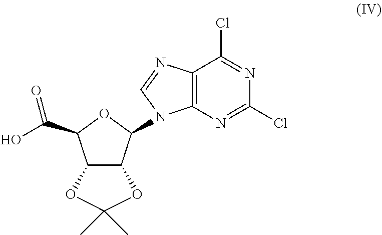 [SMILES: CC1(C)O[C@@H]2[C@H](O1)[C@@H](C(=O)O)O[C@H]2n1cnc2c(Cl)nc(Cl)nc21]

(c) chlorinating the compound of formula (IV) to obtain a compound of formula (V);

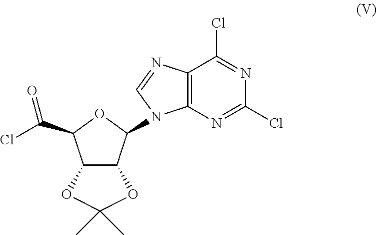 [SMILES: CC1(C)O[C@@H]2[C@H](O1)[C@@H](C(=O)Cl)O[C@H]2n1cnc2c(Cl)nc(Cl)nc21]

(d) aminating the compound of formula (V) to obtain a compound of formula (VI);

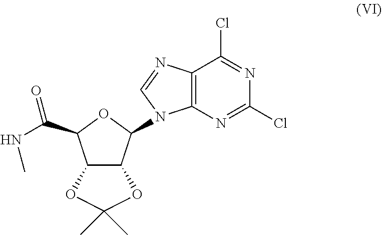 [SMILES: CNC(=O)[C@H]1O[C@@H](n2cnc3c(Cl)nc(Cl)nc32)[C@@H]2OC(C)(C)O[C@@H]21]

(e) reacting the compound of formula (VI) with the compound of formula (VII)

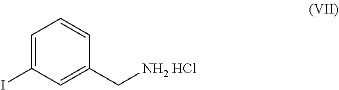

to obtain a compound of formula (VIII); and

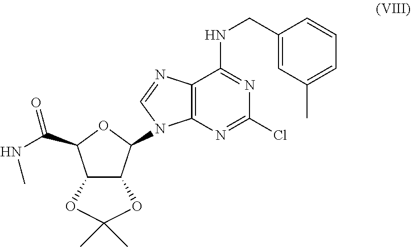 [SMILES: CNC(=O)[C@H]1O[C@@H](n2cnc3c(NCc4cccc(C)c4)nc(Cl)nc32)[C@@H]2OC(C)(C)O[C@@H]21]

(f) deprotecting the compound of formula (VIII).

The processes of the present subject matter are advantageous in that they simplify the production of namodenoson while achieving a higher yield and purity of the product, as compared to the methods known in the prior art. In addition, the process reduces the level of impurities, particularly such that may pose a health hazard.

The namodenosone produced by the process of the present disclosure can be pharmaceutically used and is scalable to production under Good Manufacturing Practice (GMP).

The term “protecting”, as used herein refers to the use of a chemical moiety which protects a reactive portion of a molecule. In chemical reactions, protecting is used to prevent side reactions in those reactive portions of the molecule, while manipulating or reacting a different portion of the molecule.

In an embodiment, the compound of formula (III) is prepared from the compound of formula (II), by protecting the diol of 2,6-dichloropurine riboside of formula (II). The protection of the diols may be achieved by reacting the compound of formula (II) with 2,2-dimethoxypropane, typically in an inert solvent, such as acetone, in the presence of a catalytic amount of an acid catalyst. In a specific embodiment, the acid catalyst may be for example, p-toluenesulfonic acid. Alternatively, hydroxy-protecting groups such as, for example, methoxymethyl, benzyloxymethyl, 2-methoxyethoxymethyl, 2-(trimethylsilyl) ethoxymethyl, t-butyl, benzyl, triphenylmethyl, 2,2,2-trichloroethyl, trimethylsilyl, tert-butyl-dimethylsilyl, tert-butyldiphenylsilyl, acetate, propionate and benzoate may be used.

The protecting takes place at a temperature of about 0-20° C. for about 10-20 hours. In a specific embodiment, the compound of formula (II) undergoes protection at a temperature of about 0-10° C. for about 16 hours. When the reaction is substantially complete, the compound of formula (III) may be isolated by conventional means, for example by filtration and drying of the filtered solids.

According to an embodiment, the oxidizing agent is a percarboxylic acid selected from the group comprising of sodium periodate or potassium periodate. In a specific embodiment, the oxidizing agent is sodium periodate.

According to an embodiment, the oxidation may be carried out in the presence of a ruthenium-containing catalyst and a solvent. The catalyst utilized in accordance with the present subject matter is ruthenium metal, inorganic ruthenium salts or organic ruthenium salts. Inorganic ruthenium salts may include but are not limited to ruthenium chloride, ruthenium bromide, ruthenium dioxide, ruthenium sulfide and ruthenium carbonate. Organic ruthenium salts may include but are not limited to ruthenium formate, ruthenium acetate, ruthenium propionate and ruthenium butyrate. In a specific embodiment, the ruthenium-containing catalyst is ruthenium chloride.

The catalyst may be present in amounts between about 0.00001 and 1.0% by weight of the reaction mixture. In an embodiment, the catalyst may be present in amounts between about 0.01 and 0.1% by weight.

Suitable solvents for use in the oxidation step include any inert solvent which is not susceptible to oxidation. The oxidation reaction can be carried out as a two-solvent system by using water together with an organic solvent. In an embodiment, the organic solvent is acetonitrile.

A phase transfer catalyst may be used in the oxidation step to accelerate the oxidation reaction. The phase transfer catalyst used in the oxidation step may include but are not limited to include tetrabutylammonium iodide, tetrabutylammonium chloride, tetrabutylammonium bromide, tetraethylammonium chloride, tetraethylammonium bromide, tetraethylammonium iodide, triethylbenzylammonium chloride, triethylbenzylammonium bromide and triethylbenzylammonium iodide. In an embodiment, the phase transfer catalyst is tetrabutylammonium iodide.

The oxidation takes place at a temperature of about 10-40° C. for about 5-15 hours. In a specific embodiment, the compound of formula (III) is oxidized at a temperature of about 15-25° C. for about 9 hours. When the reaction is substantially complete, the compound of formula (IV) may be isolated by conventional means.

The chlorination is carried out in the presence of a chlorinating agent and at least one polar aprotic solvent. The chlorinating agent may be selected from the group comprising of thionyl chloride, phosphorus oxychloride, phosgene, sulfuryl chloride, phosphorus pentachloride, triphosgene, diphosgene and oxalyl chloride. In a specific embodiment, the chlorinating agent is thionyl chloride.

The polar aprotic solvent may be selected from the group comprising of acetonitrile, methyl tert-butyl ether, tetrahydrofuran, dichloromethane, 1,2 dichloroethane, dimethylformamide, dimethylacetamide, ethyl acetate, acetone, dimethyl sulfoxide methyl iso-butyl ketone, isopropylacetate 2-ethyltetrahydrofuran, 1,4-dioxane, CPME, dimethoxyethane, diglyme and diethoxymethane. In a specific embodiment, the polar parotic solvent is acetonitrile.

The amination may be carried out in the presence of an amination agent selected from the group comprising of methyl amine, tert-butyl amine, benzyl amine, p-methoxybenzyl amine, 3,4-dimethoxybenzyl amine, allyl amine, methoxymethyl amine, triphenylmethyl amine, benzoyl amine, dinitrophenyl amine, p-methoxyphenyl amine. In a specific embodiment, the amination agent is methyl amine.

The amination reaction may be carried out in the presence of a base. Suitable bases include tertiary amines such as trimethylamine, triethylamine, diisopropylethylamine (DIPEA), pyridine, 4-dimethylaminopyridine (DMAP) and N-methylmorpholine.

According to an embodiment, the compound of formula (V) is not isolated prior to its reaction with the amination agent. According to another embodiment, the chlorination and amination are carried out in one pot, without isolating the compound of formula (V).

According to an embodiment, the compound of formula (VIII) is not isolated prior to the deprotecting step. According to another embodiment, steps (d) and (e) are carried out in one pot, without isolating the compound of formula (V).

The term “deprotecting”, as used herein refers to the removal of the chemical moiety which was used to protect a portion of the molecule by a deprotecting agent. The chemical moiety is readily displaceable by a nucleophile, for example, under nucleophilic substitution reaction conditions. In an embodiment, the deprotection agent can be an acid. The acid may be selected from but not limited to, hydrochloric acid, hydrobromic acid, sulfuric acid, phosphoric acid, acetic acid and trifluoroacetic acid. In a specific embodiment, hydrochloric acid is used to deprotect the compound of formula (VIII). In a further embodiment, deprotection the compound of formula (VIII) is carried out with an acid, such as hydrochloric acid, in a suitable solvent, such as tetrahydrofuran, to provide the diol in the compound of formula (I).

In an embodiment, after the compound of formula (VIII) is deprotected, the resultant crude compound of formula (I) is recrystallized. The crude compound of formula (I) may be recrystallized in a solution of dimethylformamide (DMF) and water.

According to an embodiment, the resultant compound of formula (I) is present at a purity of at least 80%, at least 85%, at least 88%, at least 89%, at least 90%, at least 91%, at least 92%, at least 93%, at least 94%, at least 95%, at least 96%, at least 97%, at least 98%, or at least 99%.

The progress of the reaction can be monitored using any suitable method, which can include, for example, chromatographic methods such as, e.g., high performance liquid chromatography (HPLC), thin layer chromatography (TLC), and the like.

In yet another embodiment, the compound of formula (I) can be isolated from the reaction mixture by any conventional techniques well-known in the art. Such isolation techniques can be selected, without limitation, from the group consisting of concentration, extraction, precipitation, cooling, filtration, crystallization, centrifugation, and a combination thereof, followed by drying.

In yet another embodiment, the compound of formula (I) can be optionally purified by any conventional techniques well-known in the art. Such purification techniques can be selected, without limitation, from the group consisting of precipitation, crystallization, extraction, slurrying, washing in a suitable solvent, filtration through a packed-bed column, dissolution in an appropriate solvent, re-precipitation by addition of a second solvent in which the compound is insoluble, and a combination thereof.

The following examples illustrate the practice of the present subject matter in some of its embodiments. However, they should not be construed as limiting the scope of the present subject matter. Other embodiments will be apparent to one skilled in the art from consideration of the specification and examples. It is intended that the specification, including the examples, is considered exemplary only without limiting the scope and spirit of the present subject matter.

### Preparation of Compound (III)

A reactor was charged with 218 Kg of acetone (218 kg), 69.2 kg of 2,6-Dichloropurine Riboside (Compound (II)) and 3.5 kg of p-toluenesulfonic acid. 67.5 kg of 2-Dimethoxypropane (67.5 kg) was added to the reactor while maintaining an internal temperature of 0-10° C. The reaction mixture was stirred for 6 hours. The reaction mixture was cooled to 0-10° C. and 692 kg of an 0.5% NaHCO3 aqueous solution was added while maintaining an internal temperature of 0-10° C. The mixture was stirred for 16 hours. The mixture was filtered, and the solids were washed with an acetone/water mixture. The solids were dried at 30-40° C. for 24 hours to provide the compound of formula (III).

### Preparation of Compound (IV)

A reactor was charged with 66.9 kg of the compound of formula (III), 0.67 kg of ruthenium chloride, 0.70 kg of tetrabutylammonium iodide and 790 kg of acetonitrile. The mixture was stirred at 15-25° C. until all the solids dissolved. 268 kg of water was added to the reactor, followed by 99.4 kg of sodium periodate which was added in portions. An internal temperature of 15-25° C. was maintained. The reaction mixture was stirred for 9 hours. The mixture was filtered, and the solids were washed with acetonitrile. The combined filtrate was treated with 62 kg of 20% Na₂SO₃ aq. solution, the resulting mixture was filtered, and the solids were washed with acetonitrile. The combined filtrate was treated with 4 kg of 20% Na₂SO₃ aq. solution (4 kg), filtered and washed with acetonitrile.

The combined acetonitrile solution underwent circulated filtration through CUNO (Zeta Plus™ Activated Carbon Filter) filtration over 35 hours. The combined filtrate was concentrated to 2˜3 V under reduced pressure below 40° C. and then was cooled to 15-25° C. 4 kg of 7% Na₂SO₄ aqueous solution was slowly added to the concentrate while maintaining an internal temperature of 15-25° C. The mixture was then cooled to −3 to 8° C. and allowed to stir for 12 hours. The mixture was filtered, and the filter cake was washed with water. The solids were dried at 35-43° C.

### Preparation of Compound (V)

A reactor was charged with 432 kg of acetonitrile and 40.9 kg of the compound of formula (IV). The mixture was stirred until all the solids dissolved. 17.4 kg of thionyl chloride was added while maintaining an internal temperature of 20-30° C., followed by rinse with acetonitrile. The reaction mixture was allowed to stir for 4-12 hours. A further 2.2 kg of thionyl chloride was added to drive the reaction completion.

### Preparation of Compound (VI)

The mixture obtained in the previous step was cooled to −18 to −8° C. 3.6 kg of methyl amine (8.1% wt % in THF) and 41.4 kg of N,N-diisopropylethylamine (DIPEA) were added while maintaining an internal temperature of −18 to −8° C. The reaction mixture was allowed to stir for 1-2 hour. An additional 2.0 kg of DIPEA was added, and the reaction mixture was allowed to stir for 1-2 hour. 124 kg of 10% aqueous citric acid was added to the mixture while maintaining an internal temperature of −18 to −8° C. The reaction mixture was concentrated to 3-5V under reduced pressure below 40° C. 144 kg of acetonitrile was added to the reaction mixture and concentrated to 3-5V under reduced pressure below 40° C. until all solvents were removed. The concentrate was cooled to 20-30° C. and 430 kg of 2-propyl acetate was added while maintaining an internal temperature of 20-30° C. The mixture was stirred for 1-2 hours, and subsequently allowed to stand for 0.5-1 hour. 146 kg of 2-propyl acetate was used to extract the aqueous layer from the organic layers. The combined organic layers were washed successively with 10% citric acid aqueous solution, 7% NaHCO₃ aq. and 10% Na₂SO₄ aq. The resulting organic solution was filtered through CUNO filtration for over 5 hours, and the CUNO was rinsed with 2-propyl acetate. The combined mixture was then concentrated to 3-5V under reduced pressure below 40° C. 144 kg of 2-propyl acetate to the concentrate. The mixture was again concentrated to 3-5V. A further 147 kg of 2-propyl acetate was added to the concentrate. The mixture was concentrated to 3-5V for the third time. N-heptane was then added to the concentrate while maintaining an internal temperature of 25±5° C. The mixture was cooled to 10±5° C. and stirred for 15-20 hours. The mixture was filtered, and the solids were washed with n-Heptane. The solids were dried at 35-45° C. for 16-24 hours.

### Preparation of Compound (VIII)

A reactor was charged with 366 kg of dichloromethane, 25 kg of the compound of formula (VI) and 17.4 kg of 3-iodobenzylamine hydrochloride. The mixture was stirred at 20-30° C. for 0.5-2 hours until the solids completely dissolved. 25.2 kg of diisopropylethylamine was added followed by a rinse of the mixture with 12 kg of dichloromethane, while maintaining an internal temperature of 20-30° C. The reaction mixture was allowed to stir at 30-40° C. for 25-30 hours. 150 kg of 10% aqueous citric acid was added to the mixture while maintaining an internal temperature of 10-20° C. The mixture was stirred for 3-5 hours, and the layers were separated. The organic layer was concentrated to 3-5V under reduced pressure below 40° C. To the organic layer was added 128 L of THF. The mixture was again concentrated to 3-5V under reduced pressure below 40° C. To the organic layer was added 128 L of THF and it was concentrated to 3-5V under the reduced pressure below 40° C. for the third time. To the organic layer was added a further 128 L of THF and it was concentrated to 3-5V under the reduced pressure below 40° C. for the fourth time. To the organic layer was added 277 L of THF. The organic layer was circularly filtered through CUNO for 5-10 hours. The combined filtrate was concentrated to 12-13 V under reduced pressure below 40° C. 75 litres of THF were added to the concentrate.

### Preparation of Namodenoson

To the THF solution of the compound of formula (VIII), as prepared in the previous step, 24.8 kg of 35% hydrochloric acid was added. The reaction mixture was then rinsed with 18 kg of THF, while maintaining an internal temperature of 15-25° C. The mixture was warmed to 25-35° C. and stirred for 20-30 hours. After the mixture was cooled to −5-5° C., 334 kg of 7% aqueous sodium bicarbonate was added to the mixture while maintaining an internal temperature of −5 to 5° C. at a pH of 7. The mixture was concentrated to 12-13 V under reduced pressure. To the concentrate was added 137 kg of methanol and 82 kg of THF. The mixture was heated to 35-45° C. followed by cooling to 15-25° C. The mixture was stirred at 15-25° C. for 12-16 hours and filtered. The solids were washed with methanol/water. The solids were slurred with water, filtered, and washed with water. The wet cake was re-slurred in the mixture of water/MeOH/THF, filtered and washed with methanol/water followed by drying at 35-45° C. to give crude namodenoson.

### Purification of Namodenoson (GMP Purification)

A reactor was charged with 34.6 kg of crude namodenoson, 164 kg of dimethylformamide. The mixture was stirred at 20-30° C. until all solids dissolved. The solution was filtered into a second reactor via the cartridge filter while rinsing with dimethylformamide. The mixture was warmed to 45-55° C. and stirred for 0.5-1 hour. Purified water was added to the mixture, followed by adding 0.2 kg of namodenoson crystal seed. After stirring for 16-24 hours, purified water was added over a period of 0.5-1 hour. The mixture was stirred at 45-55° C. for another 16-24 hours. The reactor was charged with 190 kg of purified water for 20-24 hours. The resulting mixture was stirred for another 5-10 hours. The mixture was filtered, and the solids were washed with water. The solids were dried at 25-35° C. for 4-8 hours without stirring, then at 45-55° C. for 18-26 hours and at 65-75° C. for 48-72 hours. After sieving, 30.65 kg of the desired final product was obtained as namodenoson drug substance.

While the present subject matter has been shown and described with reference to preferred embodiments thereof, it will be understood by those skilled in the art that many alternatives, modifications and variations may be made thereto without departing from the spirit and scope thereof. Accordingly, it is intended to embrace all such alternatives, modifications, and variations that fall within the spirit and broad scope of the claims.

All publications, patents and patent applications mentioned in this specification are herein incorporated in their entirety by reference into the specification, to the same extent as if each individual publication, patent or patent application was specifically and individually indicated to be incorporated herein by reference.

1. A process for preparing namodenoson of formula (I) 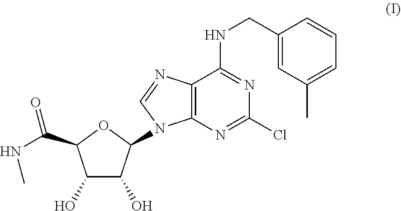 [SMILES: CNC(=O)[C@H]1O[C@@H](n2cnc3c(NCc4cccc(C)c4)nc(Cl)nc32)[C@H](O)[C@@H]1O]
comprising:
(a) protecting the hydroxyl groups of the diol of 2,6-dichloropurine riboside of formula (II) 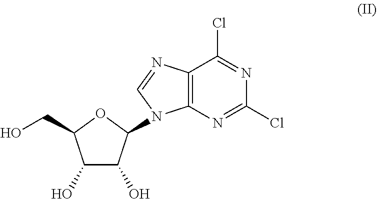 [SMILES: OC[C@H]1O[C@@H](n2cnc3c(Cl)nc(Cl)nc32)[C@H](O)[C@@H]1O]
to obtain a compound of formula (III); 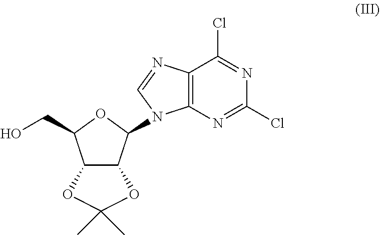 [SMILES: CC1(C)O[C@@H]2[C@H](O1)[C@@H](CO)O[C@H]2n1cnc2c(Cl)nc(Cl)nc21]
(b) oxidizing the compound of formula (III) in the presence of an oxidizing agent to obtain a compound of formula (IV); 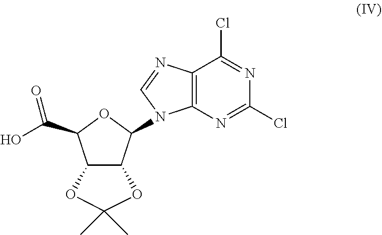 [SMILES: CC1(C)O[C@@H]2[C@H](O1)[C@@H](C(=O)O)O[C@H]2n1cnc2c(Cl)nc(Cl)nc21]
(c) chlorinating the compound of formula (IV) to obtain a compound of formula (V); 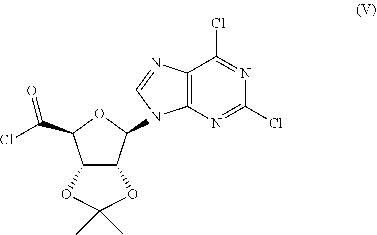 [SMILES: CC1(C)O[C@@H]2[C@H](O1)[C@@H](C(=O)Cl)O[C@H]2n1cnc2c(Cl)nc(Cl)nc21]
(d) aminating the compound of formula (V) to obtain a compound of formula (VI); 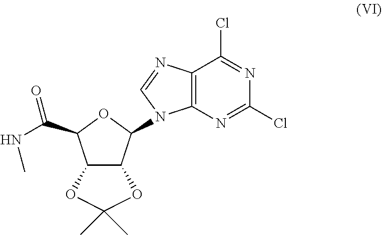 [SMILES: CNC(=O)[C@H]1O[C@@H](n2cnc3c(Cl)nc(Cl)nc32)[C@@H]2OC(C)(C)O[C@@H]21]
(e) reacting the compound of formula (VI) with the compound of formula (VII) 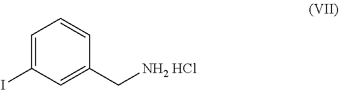
to obtain a compound of formula (VIII); and 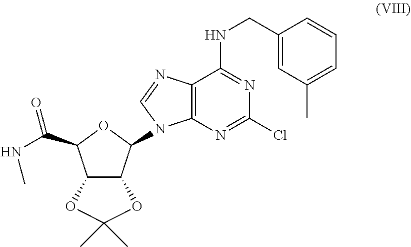 [SMILES: CNC(=O)[C@H]1O[C@@H](n2cnc3c(NCc4cccc(C)c4)nc(Cl)nc32)[C@@H]2OC(C)(C)O[C@@H]21]
(f) deprotecting the compound of formula (VIII).

2. The process of claim 1, wherein the protecting is carried out in the presence of an acid catalyst and 2-dimethylpropane.

3. The process of claim 2, wherein the acid catalyst is p-toluenesulfonic acid.

4. The process of claim 1, wherein the oxidizing agent is a percarboxylic acid.

5. The process of claim 4, wherein the percarboxylic acid is selected from sodium periodate or potassium periodate.

6. The process of claim 1, wherein the oxidizing is carried out in the presence of a ruthenium-containing catalyst and a solvent.

7. The process of claim 6, wherein the ruthenium-containing catalyst is selected from the group comprising ruthenium metal, inorganic ruthenium salts and organic ruthenium salts.

8. The process of claim 7, wherein the ruthenium-containing catalyst is ruthenium chloride.

9. The process of claim 6, wherein the solvent in the oxidizing step is a mixture of acetonitrile and water.

10. The process of claim 1, wherein the chlorination is carried out in the presence of a chlorinating agent and a polar aprotic solvent.

11. The process of claim 10, wherein the chlorinating agent is thionyl chloride.

12. The process of claim 10, wherein the polar aprotic solvent is acetonitrile.

13. The process of claim 1, wherein the amination is carried out in the presence of an amination agent.

14. The process of claim 13, wherein the amination agent is methyl amine.

15. The process of claim 1, wherein the chlorination and amination are carried out in one pot, without isolating the compound of formula (V).

16. The process of claim 1, wherein the compound of formula (VIII) is not isolated prior to the deprotecting step.

17. The process of claim 1, wherein the namodenoson of formula (I) is recrystallized.

18. The process of claim 1, wherein the resulting compound of formula (I) is present at a purity of at least 80%, at least 85%, at least 90%, at least 95%, at least 98%, or at least 99%.
